(19) United States
(12) Patent Application Publication
(10) Pub. No.: US 2006/0025420 A1
(43) Pub. Date: Feb. 2, 2006

(54) Pharmaceutical compositions for the treatment of female sexual disorders

(75) Inventors: Ulrich Brauns, Biberach (DE); Arne Froemder, Ingelheim (DE); Robert Pyke, New Fairfield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignees: Boehringer Ingelheimn International GmbH, Ingelheim (DE); Boehringer Ingelheim Pharmaceuticals, Inc., Ridgefield, CT (US)

(21) Appl. No.: 11/187,422

(22) Filed: Jul. 22, 2005

Related U.S. Application Data
(60) Provisional application No. 60/592,536, filed on Jul. 30, 2004.

(51) Int. Cl. A61K 31/519; A61K 31/496

(52) U.S. Cl. 514/252.16; 514/254.06

(57) ABSTRACT

The invention relates to a method for the treatment of female sexual disorders comprising administration of a therapeutically effective amount of a compound of general formula 1 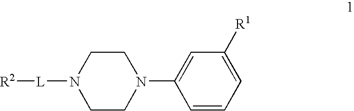 wherein the groups R, L and X may have the meanings specified in the description and claims.

The invention relates to a method for the treatment of female sexual disorders comprising administration of a therapeutically effective amount of a compound of general formula 1 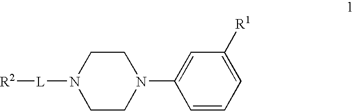
wherein the groups R, L and X may have the meanings specified in the description and claims.

### DESCRIPTION OF THE INVENTION

The invention relates to a method for the treatment of female sexual disorders comprising administration of a therapeutically effective amount of a compound of general formula 1 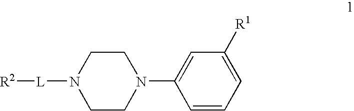
wherein
R¹ is a group selected from among halogen, —O—C₁-C₄-alkyl, and —C(halogen)₃;
L is a linker, selected from the bridging groups —C₁-C₆-alkylene, —C₁-C₄-alkylene-O—, —C₁-C₄-alkylene-O—CO—, —C₁-C₄-alkylene-NH—, —C₁-C₄-alkylene-NH—CO—, —C₂-C₆-alkenylene, —C₂-C₄-alkenylene-O—, —C₂-C₄-alkenylene-O—CO—, —C₂-C₄-alkenylene-NH—, —C₂-C₄-alkenylene-NH—CO—, —C₂-C₆-alkynylene, —C₂-C₄-alkynylene-O—, —C₂-C₄-alkynylene-O—CO—, —C₂-C₄-alkynylene-NH—, and —C₂-C₄-alkynylene-NH—CO—, which may optionally be substituted by one or more, preferably one group selected from among —C₁-C₄-alkyl, —OH, halogen, ═O, —C(halogen)₃ and —O—C₁-C₄-alkyl;
R² is —NH₂, —NHC₁-C₄-alkyl, —N(C₁-C₄-alkyl)₂, or a group selected from among —C₁-C₆-alkyl and —C₃-C₆-cycloalkyl which may optionally be substituted by one or more, preferably one group selected from among —C₁-C₄-alkyl, —OH, halogen, ═O, —C(halogen)₃, —O—C₁-C₄-alkyl, —O—C₆-C₁₀-aryl, —NH₂, —NHC₁-C₄-alkyl, —N(C₁-C₄-alkyl)₂, —C₂-C₄-alkenyl and —C₂-C₄-alkynyl, or
R² is —C₆-C₁₀-aryl, optionally substituted by one or more, preferably one group selected from among, —C₁-C₄-alkyl, —OH, halogen, —C(halogen)₃, —O—C₁-C₄-alkyl, —NH₂, —NH—C₁-C₄-alkyl, —N(C₁-C₄-alkyl)₂, and a nitrogen containing heteroaromatic ring, wherein said nitrogen containing heteroaromatic ring may optionally be substituted by one or more, preferably one group selected from among —C₁-C₄-alkyl, —OH, halogen, —C(halogen)₃, and —O—C₁-C₄-alkyl, and wherein said nitrogen containing heteroaromatic ring my optionally be linked to the —C₆-C₁₀-aryl group via a bridging group selected from among —O—, —S—, and —NH—, or R² is a group selected from among 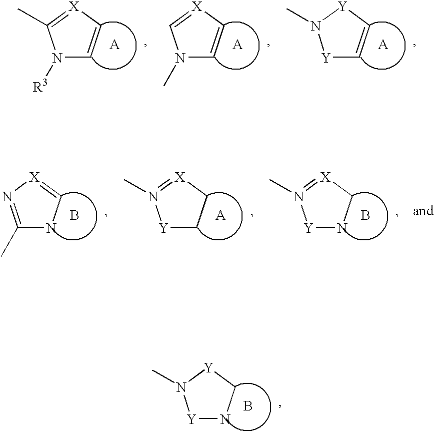
   wherein
   X is either N or —CR³—;
   Y is either —NR⁵—, —O—, —S—, —SO₂—, —CH₂— or —CO—;
   A is absent or a ring system selected from among 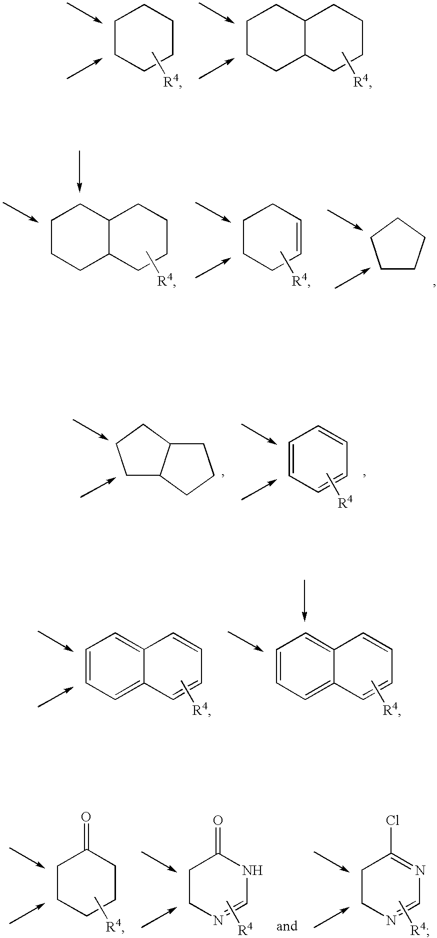
   B is absent or a ring system selected from among 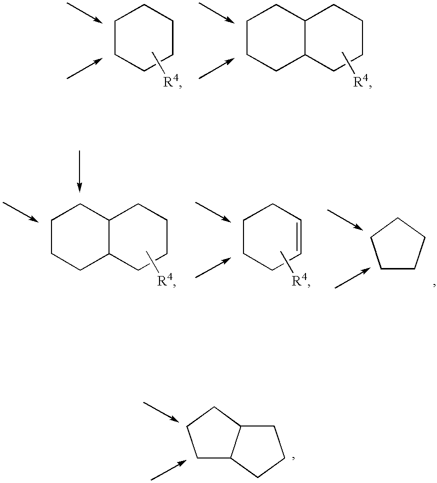
   wherein the arrows indicate the positions where the ring is annellated to the five membered nitrogen heterocycle, and wherein
   R³ is selected from among hydrogen, —C₁-C₄-alkyl, —CH₂—NH₂, —CH₂—NH—C₁-C₄-alkyl, —CH₂—N(C₁-C₄-alkyl)₂, —NH₂, —NH—C₁-C₄-alkyl, and
   —N(C₁-C₄-alkyl)₂;
   R⁴ is selected from among hydrogen, —C₁-C₄-alkyl, —OH, halogen, —C(halogen)₃ and —O—C₁-C₄-alkyl,
   R⁵ is selected from among hydrogen, —C₁-C₄-alkyl, —C₆-C₁₀-aryl, and —C₁-C₄-alkylen-C₆-C₁₀-aryl;
      optionally in form of the pharmaceutically acceptable acid addition salts, in form of the hydrates and/or solvates and optionally in the form of the individual optical isomers, mixtures of the individual enantiomers or racemates thereof.

In a preferred embodiment the invention relates to a method for the treatment of female sexual disorders selected from the group consisting of Hypoactive Sexual Desire Disorder, loss of sexual desire, lack of sexual desire, decreased sexual desire, inhibited sexual desire, loss of libido, libido disturbance, and frigidity, comprising the administration of a therapeutically effective amount of a compound of formula 1 optionally in form of the pharmaceutically acceptable acid addition salts, in form of the hydrates and/or solvates and optionally in the form of the individual optical isomers, mixtures of the individual enantiomers or racemates thereof. In another preferred embodiment the invention relates to a method for the treatment of disorders selected from the group consisting of Hypoactive Sexual Desire Disorder, loss of sexual desire, lack of sexual desire, decreased sexual desire, inhibited sexual desire, comprising the administration of a therapeutically effective amount of a compound of formula 1 optionally in form of the pharmaceutically acceptable acid addition salts, in form of the hydrates and/or solvates and optionally in the form of the individual optical isomers, mixtures of the individual enantiomers or racemates thereof. In another preferred embodiment the invention relates to a method for the treatment of disorders selected from the group consisting of Hypoactive Sexual Desire Disorder and loss of sexual desire, preferably Hypoactive Sexual Desire Disorder, comprising the administration of a therapeutically effective amount of a compound of formula 1 optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of premenstrual disorders, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of premenstrual disorders, selected from the group consisting of premenstrual dysphoria, premenstrual syndrome and premenstrual dysphoric disorder, in particular premenstrual dysphoric disorder, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of sexual aversion disorder in females, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of sexual arousal disorder in females, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of orgasmic disorder in females, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of sexual pain disorders in females, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

In a particular preferred embodiment the invention relates to a method for the treatment sexual pain disorders selected from the group consisting of dyspareunia, vaginismus, noncoital sexual pain disorder, sexual dysfunction due to a general medical condition and substance-induced sexual dysfunction, comprising the administration of a therapeutically effective amount of a compound of formula 1, optionally in form of the pharmaceutically acceptable acid addition salts, in form of the hydrates and/or solvates and optionally in the form of the individual optical isomers, mixtures of the individual enantiomers or racemates thereof.

The beneficial effects of the compositions according to the invention can be observed regardless of whether the disturbance existed lifelong or was acquired, and independent of etiologic origin (organic—both, physically and drug induced-, psychogen, a combination of organic—both, physically and drug induced-, and psychogen, or unknown).

Another embodiment of the invention relates to the use of the compounds of formula 1, optionally in form of the pharmaceutically acceptable acid addition salts, in form of the hydrates and/or solvates and optionally in the form of the individual optical isomers, mixtures of the individual enantiomers or racemates thereof for the preparation of a medicament for the treatment of the aforementioned disorders.

In a preferred embodiment the invention relates to a method for the treatment of the disorders mentioned hereinbefore, comprising administration of a therapeutically effective amount of a compound of general formula 1, wherein
R¹ is a group selected from among fluorine, chlorine, —O-methyl, and —CF₃, preferably chlorine and —CF₃;
L is a linker, selected from the bridging groups —C₁-C₄-alkylene, —C₁-C₃-alkylene-O—, —C₁-C₃-alkylene-O—CO—, —C₁-C₃-alkylene-NH—, —C₁-C₃-alkylene-NH—CO—, and —C₂-C₄-alkenylene, which may optionally be substituted by one or more, preferably one group selected from among methyl, ethyl, propyl, —OH, chlorine, fluorine, =0 and —CF₃;
R² is —NH₂, —NHC₁-C₄-alkyl, —N(C₁-C₄-alkyl)₂, or a group selected from among —C₁-C₄-alkyl and —C₃-C₆-cycloalkyl which may optionally be substituted by one or more, preferably one group selected from among —OH, fluorine, chlorine, ═O, —CF₃, —O-phenyl, —O-naphthyl, —NH₂, —NHmethyl, —N(methyl)₂, —C₂-C₄-alkenyl and —C₂-C₄-alkynyl, or R² is a phenyl or naphthyl group, optionally substituted by one or more, preferably one group selected from among, methyl, ethyl, —OH, fluorine, chlorine, —CF₃, —O-methyl, —O-ethyl, —NH₂, —NH-methyl, —N(methyl)₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, ethyl, —OH, fluorine, chlorine, —CF₃, —O-methyl and —O-ethyl, and wherein said nitrogen containing heteroaromatic ring my optionally be linked to the phenyl or naphthyl group via a bridging group selected from among —O— and —NH—, or
R² is a group selected from among 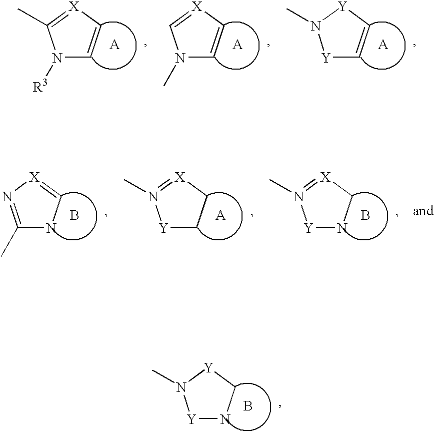
   wherein
   X is either N or —CR³—;
   Y is either —NR⁵—, —O—, or —CO—;
   A is absent or a ring system selected from among 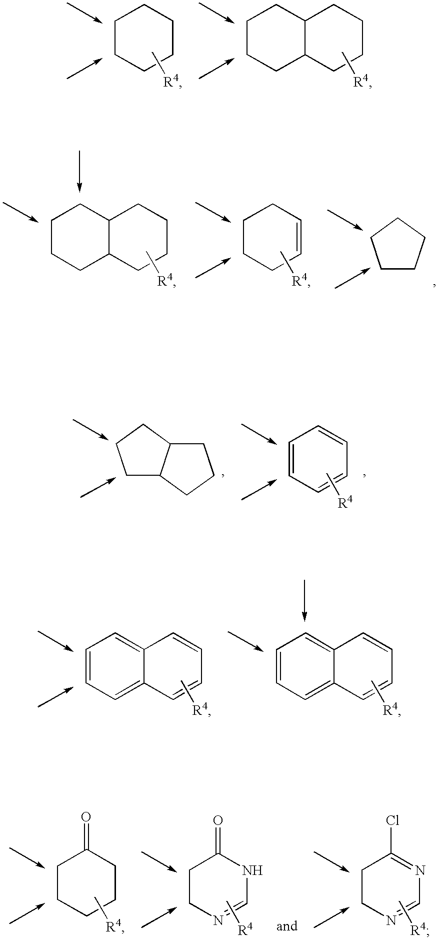
   B is absent or a ring system selected from among 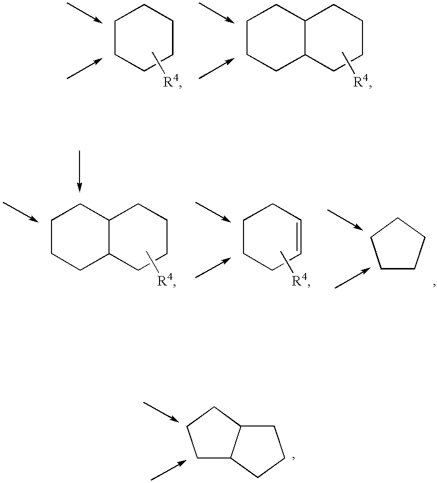
   wherein the arrows indicate the positions where the ring is annellated to the five membered nitrogen heterocycle, and wherein
   R³ is selected from among hydrogen, methyl, ethyl, propyl, —CH₂—N(methyl)₂, and —N(methyl)₂;
   R⁴ is selected from among hydrogen, methyl, ethyl, propyl, —OH, chlorine, fluorine and —CF₃,
   R⁵ is selected from among hydrogen, methyl, ethyl, propyl, phenyl, —CH₂—CH₂-phenyl and Benzyl; optionally in form of the pharmaceutically acceptable acid addition salts, in form of the hydrates and/or solvates and optionally in the form of the individual optical isomers, mixtures of the individual enantiomers or racemates thereof.

In a preferred embodiment the invention relates to a method for the treatment of the disorders mentioned hereinbefore, comprising administration of a therapeutically effective amount of a compound of general formula 1, wherein
R¹ is a group selected from among fluorine, chlorine, —O-methyl, and —CF₃, preferably chlorine and —CF₃;
L is a linker, selected from the bridging groups —C₁-C₄-alkylene, —C₁-C₃-alkylene-O—, —C₁-C₃-alkylene-O—CO—, —C₁-C₃-alkylene-NH—, —C₁-C₃-alkylene-NH—CO—, and —C₂-C₄-alkenylene, which may optionally be substituted by one or more, preferably one group selected from among methyl, ethyl, propyl, —OH, chlorine, fluorine, =0 and —CF₃;
R² is —NH₂, —NHC₁-C₄-alkyl, —N(C₁-C₄-alkyl)₂, or a group selected from among —C₁-C₄-alkyl and —C₃-C₆-cycloalkyl which may optionally be substituted by one or more, preferably one group selected from among —OH, fluorine, chlorine, ═O, —CF₃, —O-phenyl, —O-naphthyl, —NH₂, —NHmethyl, —N(methyl)₂, —C₂-C₄-alkenyl and —C₂-C₄-alkynyl, or
R² is a phenyl or naphthyl group, optionally substituted by one or more, preferably one group selected from among, methyl, ethyl, —OH, fluorine, chlorine, —CF₃, —O-methyl, —O-ethyl, —NH₂, —NH-methyl, —N(methyl)₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, ethyl, —OH, fluorine, chlorine, —CF₃, —O-methyl and —O-ethyl, and wherein said nitrogen containing heteroaromatic ring my optionally be linked to the phenyl or naphthyl group via a bridging group selected from among —O— and —NH—, or
R² is a group selected from among 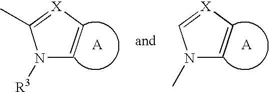
   wherein
   X is either N or —CR³—;
   R³ is selected from among hydrogen, methyl, ethyl, propyl, —CH₂—N(methyl)₂, and —N(methyl)₂;
   A is a absent or a ring system selected from among 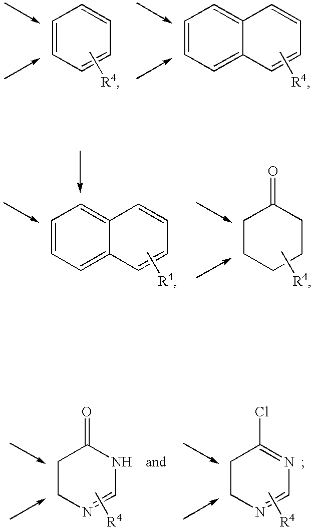
      wherein the arrows indicate the positions where the ring is annellated to the five membered nitrogen heterocycle, and wherein
      R⁴ is selected from among hydrogen, methyl, ethyl, propyl, —OH, chlorine, fluorine and —CF₃,
         optionally in form of the pharmaceutically acceptable acid addition salts, in form of the hydrates and/or solvates and optionally in the form of the individual optical isomers, mixtures of the individual enantiomers or racemates thereof.

In a preferred embodiment the invention relates to a method for the treatment of the disorders mentioned hereinbefore, comprising administration of a therapeutically effective amount of a compound of general formula 1, wherein
R¹ is a group selected from among chlorine and —CF₃, preferably —CF₃;
L is a linker, selected from —CH₂—CH₂—, —CH₂—CH₂—CH₂—, —CH₂—CH₂—CH₂—CH₂—, —O—CH₂—CH₂—, —O—CH₂—CH₂—CH₂—, —O—CH₂—CH₂—CH₂—CH₂—, —CO—O—CH₂—CH₂—, —CO—O—CH₂—CH₂—CH₂—, —CO—O—CH₂—CH₂—CH₂—CH₂—, —N H—CH₂—CH₂—, —NH—CH₂—CH₂—CH₂—, —NH—CH₂—CH₂—CH₂—CH₂—, —CO—NH—CH₂—CH₂—, —CO—NH—CH₂—CH₂—CH₂— and —CO—NH—CH₂—CH₂—CH₂—CH₂—, which may optionally be substituted by one or more, preferably one group selected from among methyl, —OH, fluorine, and —CF₃;
R² is —NH₂, —NHC, —C₄-alkyl, —N(C₁-C₄-alkyl)₂, or a group selected from among methyl and ethyl which may optionally be substituted by one or more, preferably one group selected from among —OH, fluorine, chlorine, ═O, —CF₃, —O-phenyl, and —NH₂, or
R² is a group selected from among cyclopentyl and cyclohexyl, which may optionally be substituted by one or more, preferably one group selected from among —OH, fluorine, —CF₃, and —C≡C—, or
R² is a phenyl group, optionally substituted by one or more, preferably one group selected from among, methyl, —OH, fluorine, —CF₃, —NH₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, —OH, fluorine, and
   —CF₃, and wherein said nitrogen containing heteroaromatic ring my optionally be linked to the phenyl group via a bridging group selected from among —O— and —NH—, or
R² is a group selected from among 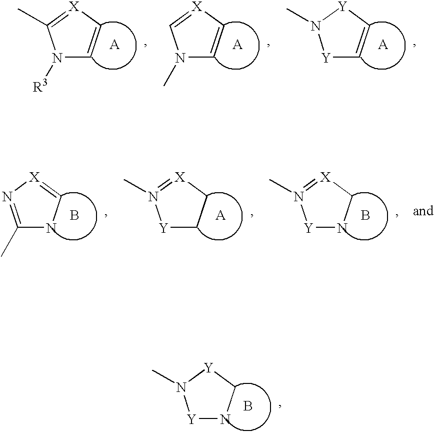
   wherein
   X is either N or —CH—;
   Y is —O— or —CO—;
   A is absent or a ring system selected from among 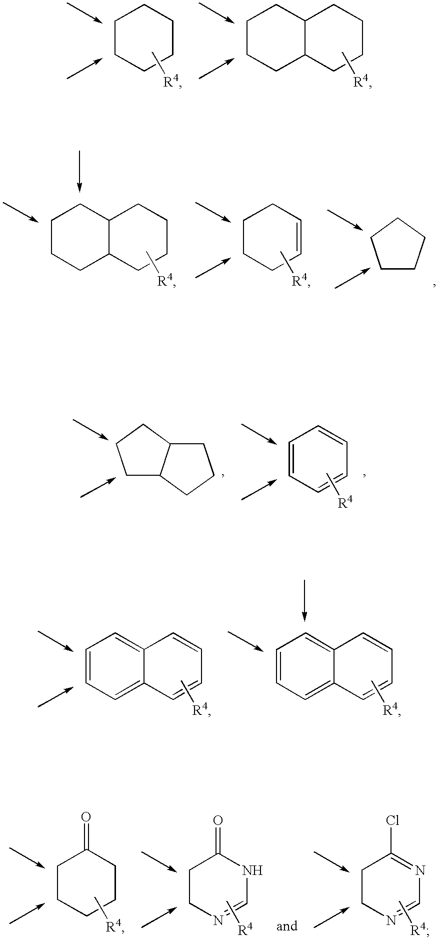
   B is absent or a ring system selected from among 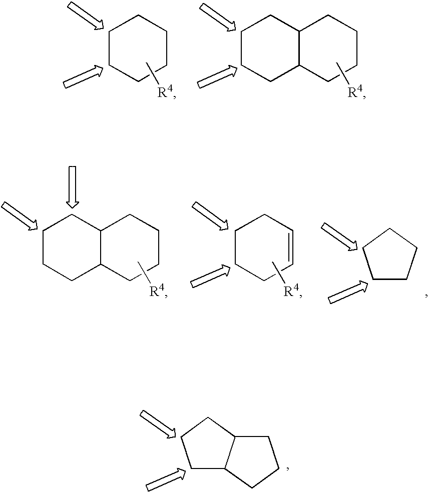
   wherein the arrows indicate the positions where the ring is annellated to the five membered nitrogen heterocycle, and wherein
   R⁴ is selected from among hydrogen, methyl, —OH, fluorine and —CF₃,
      optionally in form of the pharmaceutically acceptable acid addition salts, in form of the hydrates and/or solvates and optionally in the form of the individual optical isomers, mixtures of the individual enantiomers or racemates thereof.

In a preferred embodiment the invention relates to a method for the treatment of the disorders mentioned hereinbefore, comprising administration of a therapeutically effective amount of a compound of general formula 1, wherein
R¹ is a group selected from among chlorine and —CF₃, preferably —CF₃;
L is a linker, selected from —CH₂—CH₂—, —CH₂—CH₂—CH₂—, —CH₂—CH₂—CH₂—CH₂—, —O—CH₂—CH₂—, —O—CH₂—CH₂—CH₂—, —O—CH₂—CH₂—CH₂—CH₂—, —CO—O—CH₂—CH₂—, —CO—O—CH₂—CH₂—CH₂—, —CO—O—CH₂—CH₂—CH₂—CH₂—, —N H—CH₂—CH₂—, —NH—CH₂—CH₂—CH₂—, —NH—CH₂—CH₂—CH₂—CH₂—, —CO—NH—CH₂—CH₂—, —CO—NH—CH₂—CH₂—CH₂— and —CO—NH—CH₂—CH₂—CH₂—CH₂—, which may optionally be substituted by one or more, preferably one group selected from among methyl, —OH, fluorine, and —CF₃;
R² is —NH₂, —NHC₁-C₄-alkyl, —N(C₁-C₄-alkyl)₂, or a group selected from among methyl and ethyl which may optionally be substituted by one or more, preferably one group selected from among —OH, fluorine, chlorine, ═O, —CF₃, —O-phenyl, and —NH₂, or
R² is a group selected from among cyclopentyl and cyclohexyl, which may optionally be substituted by one or more, preferably one group selected from among —OH, fluorine, —CF₃, and —C≡C—, or
R² is a phenyl group, optionally substituted by one or more, preferably one group selected from among, methyl, —OH, fluorine, —CF₃, —NH₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, —OH, fluorine, and
   —CF₃, and wherein said nitrogen containing heteroaromatic ring my optionally be linked to the phenyl group via a bridging group selected from among —O— and —NH—, or
R² is a group selected from among 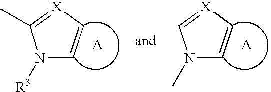
   wherein
   X is either N or —CH—;
   A is a ring system selected from among 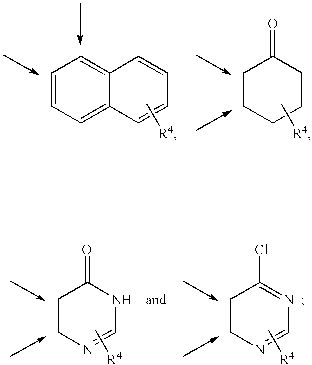
   wherein the arrows indicate the positions where the ring is annellated to the five membered nitrogen heterocycle, and wherein
   R⁴ is selected from among hydrogen, methyl, —OH, fluorine and —CF₃,
      optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein R is
—CF₃ and wherein L and R have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
L is a linker, selected from —CH₂—CH₂—, —CH₂—CH₂—CH₂—, —CH₂—CH₂—CH₂—CH₂—, —O—CH₂—CH₂—, —O—CH₂—CH₂—CH₂—, —CO—O—CH₂—CH₂—, —CO—O—CH₂—CH₂—CH₂—, —NH—CH₂—CH₂—, —NH—CH₂—CH₂—CH₂—, —CO—NH—CH₂—CH₂—,
   —CO—NH—CH₂—CH₂—CH₂— and —CO—NH—CH₂—CH₂—CH₂—CH₂—, which may optionally be substituted by one or more, preferably one group selected from among methyl, —OH, fluorine, and —CF₃,
      and wherein R¹ and R have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
L is a linker, selected from —CH₂—CH₂—, —CH₂—CH₂—CH₂—, —CH₂—CH₂—CH₂—CH₂—, —O—CH₂—CHOH—CH₂—, —CO—O—CH₂—CH₂—, —CO—NH—CH₂—CH₂—, —CO—NH—CH₂—CH₂—CH₂— and —CO—NH—CH₂—CH₂—CH₂—CH₂—,
   and wherein R¹ and R have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is —NH₂, —NHmethyl, —N(methyl)₂, or a group selected from among
   methyl and ethyl which may optionally be substituted by one or more,
   preferably one group selected from among fluorine, —CF₃, and —O-phenyl; and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is —NH₂, methyl, ethyl or —CH₂—O-phenyl;
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is a group selected from among cyclopentyl and cyclohexyl, which may optionally be substituted by one or more, preferably one group selected from among —OH, fluorine, —CF₃, and —C≡C—, or
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is cyclohexyl, which may optionally be substituted by one group selected from among —OH, fluorine, —CF₃, and —C≡C—, preferably —C≡C— or
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is a phenyl group, optionally substituted by one or more, preferably one group selected from among, methyl, —OH, fluorine, —CF₃, —NH₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, —OH, fluorine, and —CF₃, and wherein said nitrogen containing heteroaromatic ring my optionally be linked to the phenyl group via a bridging group selected from among —O— and —NH—,
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is a phenyl group, optionally substituted by one or more, preferably one group selected from among, methyl, —OH, fluorine, —CF₃ and —NH₂, preferably NH₂,
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is a phenyl group substituted by nitrogen containing heteroaromatic ring selected from among pyridine, pyrimidine, indol, pyrrole, imidazole, pyrazole, chinoline and isochinoline, wherein said nitrogen containing heteroaromatic ring may optionally be substituted by one or more, preferably one group selected from among methyl, —OH, fluorine, and —CF₃, and wherein said nitrogen containing heteroaromatic ring my optionally be linked to the phenyl group via a bridging group selected from among —O— and —NH—,
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is phenyl substituted via the bridging group —NH— by a group selected from among pyridine, pyrimidine, indol, pyrrole, imidazole, pyrazole, chinoline and isochinoline, preferably pyridine, pyrimidine, chinoline and isochinoline, which may optionally be substituted by one group selected from among methyl, —OH, fluorine, and —CF₃, preferably —CF₃,
   and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is the group 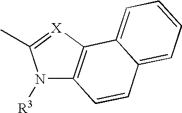
   wherein
   X is either N or —CH—;
   R³ is selected from among hydrogen, isopropyl and —CH₂—N(methyl)₂, preferably hydrogen or isopropyl,
      and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

In another preferred embodiment the invention relates to a method for the treatment of the disorders mentioned hereinbefore, comprising administration of a therapeutically effective amount of a compound of general formula 1, wherein
R² is a group selected from among 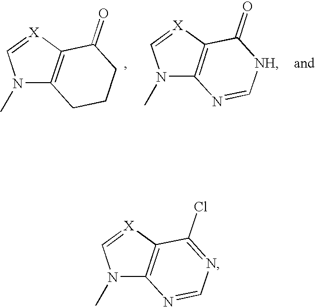
   wherein
   X is either N or —CH—,
      and wherein L and R¹ have the meanings mentioned hereinbefore or below, optionally in form of the pharmaceutically acceptable acid addition salts, in form of the hydrates and/or solvates and optionally in the form of the individual optical isomers, mixtures of the individual enantiomers or racemates thereof.

Preferred examples of compounds of formula 1 include 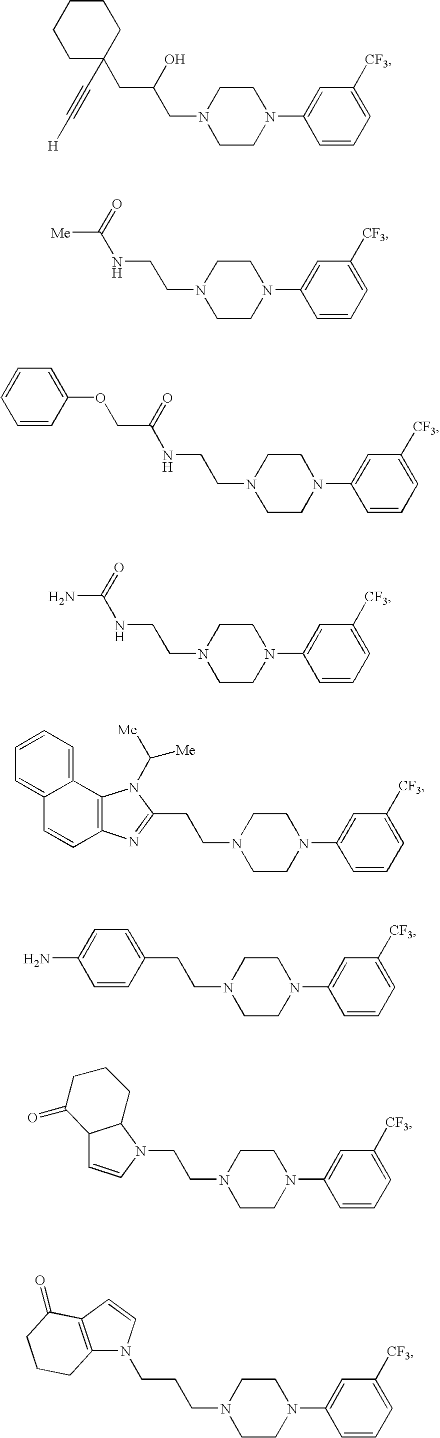 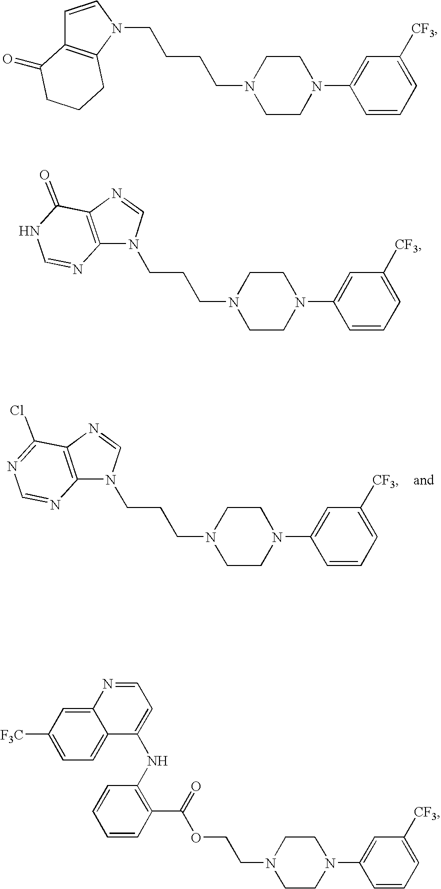
optionally in form of the pharmaceutically acceptable acid addition salts, in form of the hydrates and/or solvates and optionally in the form of the individual optical isomers, mixtures of the individual enantiomers or racemates thereof.

The alkyl groups meant here (including those which are components of other groups) are branched and unbranched alkyl groups having 1 to 6 carbon atoms, preferably 1 to 4 carbon atoms, such as: methyl (=Me), ethyl (=Et), n-propyl, iso-propyl, butyl, iso-butyl, sec.-butyl, tert.-butyl, pentyl, iso-pentyl, hexyl, heptyl and octyl.

Unless otherwise specified alkyl groups (including those which are components of other groups) may, for example, carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Alkylene groups (including those which are components of other groups) are branched and unbranched bridging groups having 1 to 6 carbon atoms, preferably 1 to 4 carbon atoms, such as: methylen, ethylen, n-propylen etc. Unless otherwise specified alkylene groups (including those which are components of other groups) may, for example, carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Alkenyl groups (including those which are components of other groups) are the branched and unbranched alkenyl groups with 2 to 6 carbon atoms, preferably 2 to 3 carbon atoms, provided that they have at least one double bond, e.g. the alkyl groups mentioned above provided that they have at least one double bond, such as for example vinyl (provided that no unstable enamines or enolethers are formed), propenyl, iso-propenyl, butenyl, pentenyl and hexenyl.

Unless otherwise specified, alkenyl groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Alkenylene groups (including those which are components of other groups) are the branched and unbranched bridging groups with 2 to 6 carbon atoms, preferably 2 to 3 carbon atoms, provided that they have at least one double bond, e.g. the alkylene groups mentioned above provided that they have at least one double bond, such as for example vinylene, propenylene, etc.

Unless otherwise specified, alkenylene groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

The term alkynyl groups (including those which are components of other groups) refers to alkynyl groups having 2 to 6 carbon atoms provided that they have at least one triple bond, e.g. ethynyl, propargyl, butynyl, pentynyl and hexynyl. Unless otherwise specified, alkynyl groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

The term alkynylene groups (including those which are components of other groups) refers to bridging groups having 2 to 6 carbon atoms provided that they have at least one triple bond, e.g. ethynylene, propargylene, etc. Unless otherwise specified, alkynylene groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Examples of cycloalkyl groups having 3 to 6 carbon atoms include cyclopropyl, cyclobutyl, cyclopentyl and cyclohexyl, which may also be substituted by branched or unbranched C₁₋₄-alkyl, hydroxy and/or halogen or as hereinbefore defined. The term halogen generally refers to fluorine, chlorine, bromine or iodine.

The word aryl denotes an aromatic ring system having 6 to 10 carbon atoms which, unless otherwise specified, may carry one or more of the following substituents, for example: C₁₋₆-alkyl, C₁₋₆-alkyloxy, halogen, hydroxy, mercapto, amino, alkylamino, dialkylamino, CF₃, cyano, nitro, —CHO, —COOH, —COO—C₁₋₆-alkyl, —S—C₁₋₆-alkyl. The preferred aryl group is phenyl.

═O denotes a double bonded oxygen atom. The term —CO—, mostly part of another group, specifies a carbonyl group. The term annellated ring is to be understood as a ring being fused to another ring fragment via two common carbon centers.

The compounds of formula 1 are capable of forming acid addition salts with pharmaceutically acceptable acids. Representative pharmaceutically acceptable acid addition salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

The compounds of formula 1 including methods for the preparation thereof are known in the art. They can be obtained for instance according to the methods described in WO 03/011396 or in analogy to the methods disclosed therein.

The term “therapeutically effective amount” shall mean that amount of a drug or pharmaceutical agent that will elicit the biological or medical response of a human that is being sought by a researcher or clinician.

The compounds 1 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

Furthermore, the invention relates to new pharmaceutical compositions for the treatment of the disorders specified hereinbefore comprising a compound of formula 1. The pharmaceutical compositions comprising a compound of formula 1 may be administered by oral, parenteral (e.g., intramuscular, intraperitoneal, intravenous or subcutaneous injection, or implant), buccal, nasal, vaginal, rectal, sublingual, or topical (e.a. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the compounds of formula 1 of this invention may conveniently be presented in dosage unit form and

may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the compounds of formula 1 that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hardgelatin or HPMC capsules wherein the active ingredient compound of formula 1 is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the compounds of formula 1 in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example

heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the compounds of formula 1 in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the compounds of formula 1 in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing compounds of formula 1 may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

Preparations according to this invention containing compounds of formula 1 for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing compounds of formula 1 may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the compounds of formula 1 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly. Within the instant invention compounds of formula 1 are preferably administered in such an amount that per single dosage between 0,001 to 1000 mg of 1 are applied.

In another embodiment, the instant invention is directed to pharmaceutical compositions comprising a therapeutically effective amount of a compound of formula 1 in combination with a therapeutically effective amount of another active ingredient for the treatment of the disorders mentioned hereinbefore.

A preferred embodiment of the invention is directed to pharmaceutical compositions comprising a therapeutically effective amount of 1 in combination with a therapeutically effective amount of one or more, preferably one active ingredient selected from the group consisting of melanocortin agonists, prostaglandin E1 agonists, elevators of cyclic guanosine 3′,5′-monophosphate (cGMP) (preferably PDE V inhibitors), 5-HT-1A agonists, dopamine agonists, dopamine D4 antagonist and 5-HT-2A/C antagonists.

The compositions according to the invention may contain 1 and the one or more additional active ingredient in a single formulation or in separate formulations. If 1 and the one or more additional active ingredient are present in separate formulations these separate formulations may be administered simultaneously or sequentially.

A preferred embodiment according to the invention is directed to pharmaceutical compositions comprising a therapeutically effective amount of 1 and a therapeutically effective amount of one or more, preferably one melanocortin agonist, optionally in combination with a pharmaceutically acceptable excipient.

Examples of suitable melanocortin agonists include PT-141, MCL-0129, PG-917, and Ro-27-3225, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one prostaglandin E1 agonist, optionally in combination with a pharmaceutically acceptable excipient. Examples of suitable prostaglandin E1 agonists, include ornoprostil, limaprost, alprostadil, gemeprost, liprostin, NMI-775, prostaglandin E (PGE-1), papaverine, dioxyline, ethaverine, phentolamine, prazosin, minoxidil, nitroglycerin, alpha blockers, nitric oxide donors, and peptides (e.g., VIP), from which ornoprostil, limaprost and alprostadil are particularly preferred optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one elevator of cGMP, preferably a cGMP phosphodiesterase (cGMP PDE) inhibitor, more preferably a selective PDE V inhibitor, optionally in combination with a pharmaceutically acceptable excipient. Examples of elevators of cGMP, in particular examples for suitable PDE V inhibitors include vardenafil, sildenafil, tadalafil, NCX-911, Sch-444877, FR-229934, 4-bromo-5-(pyridylmethylamino)-6-[3-(4-chlorophnyl)-propoxy]-3 (2H)pyridazinone, 1-[4-(1,3-benzodioxol-5-ylmethyl)amiono]-6-chloro-2-[quinozolinyl]-4-piperidine-carboxylic acid, monosodium salt, (+)-cis-5,6a,7,9,9,9a-hexahydro-2-[4-(trifluoromethyl)-phenylmethyl-5-methyl-cyclopent-4,5]imidazo[2,1-b]purin-4 (3H)one, furaziocillin, cis-2-hexyl-5-methyl-3,4,5,6a,7,8,9,9a-octahydrocyclopent[4,5]-imidazo[2,1-b]purin-4-one, 3-acetyl-1-(2-chlorobenzyl)-2 propylindole-6-carboxylate, 3-acetyl-1-(2-chlorobenzyl)-2-propylindole-6-carboxylate, 4-bromo-5-(3-pyridyl methylamino)-6-(3-(4-chlorophenyl) propoxy)-3-(2H)pyridazinone, 1-methyl-5(5-morpholinoacetyl-2-n-propoxyphenyl)-3-n-propyl-1,6-dihydro-7H-pyrazolo(4,3-d)pyrimidin-7-one, 1-[4-[(1,3-benzodioxol-5-ylmethyl)amino]-6-chloro-2-quinazolinyl}-4-piperidinecarboxylic acid, monosodium salt, GF-196960, E-8010, E-4010, Bay-38-3045, Bay-38-9456, FR226807, Sch-51866, 5-(2-ethoxy-5-morpholinoacetylphenyl)-1-methyl-3-n-propyl-1,6-dihydro-7H-pyrazolo[4,3-d]pyrimidin-7-one, 3-ethyl-5-[5-(4-ethylpiperazin-1-ylsulfonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulfonyl)-2-(2methoxyethoxy)pyridin-3-yl]-2-(pyridin-2-yl)methyl-2,6-dihydro-7H-pyrazolo[4,3-d]pyrimidin-7-one, (+)-3-ethyl-5-[5-(4-ethylpieperazin-1-ylsulfonyl)-2-(2-methoxy-1 (R)-methylethoxy)pyridin-3-yl]-2-methyl-2,6-dihydro-7H-pyrazolo[4,3-d]pyrimidin-7-one, 5-[2-ethoxy-5-(4-ethylpiperazin-1-ylsulfonyl)pyridin-3-yl]-3-ethyl-2-[2-methoxyethyl]-2,6-dihydro-7H-pyrazolo[4,3-d]pyrimidin-7-one, 5-[2-iso-butoxy-5-(4-ethylpiperazin-1-ylsulfonyl)pyridin-3-yl]-3-ethyl-2-(1-methylpiperidin-4-yl)-2,6-dihydro-7H-pyrazolo[4,3-d]pyrimidin-7-one,
5-[2-ethoxy-5-(4-ethylpiperazin-1-ylsulfonyl)pyridin-3-yl]-3-ethyl-2-phenyl-2,6-dihydro-7H-pyrazolo[4,3-d]pyrimidin-7-one, 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, 4-(4-chlorobenzyl)amino-6,7,8-trimethoxyquinazoline,
7,8-dihydro-8-oxo-6-[2-propoxyphenyl]-1H-imidazo[4,5-g]quinazoline, 1-[3-[1-[(4-fluorophenyl)methyl]-7,8-dihydro-8-oxo-1H-imidazo[4,5-g]quinazolin-6-yl]-4-propoxyphenyl]carboxamide, 2-[2-ethoxy-5-(4-ethyl-piperazine-1-sulfonyl)-phenyl]-5-methyl-7-propyl-3H-imidazo[5,1-f]-[1,2,4]-triazin-4-one, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one 5-HT-1A agonist, optionally in combination with a pharmaceutically acceptable excipient. Examples of suitable 5-HT-1A agonists include Urapidil, Buspirone, Aripiprazole, Ziprasidone, Naftopidil, Tandospirone, Nemonapride, Gepirone, Repinotan, Sumanirole, Xaliproden hydrochloride, Bifeprunox, AP-521, SUN-N4057, Sarizotan, MKC-242, OPC-14523, Eptapirone maleate, SLV-308, BTS-79018, R-137696, F-13640, SSR-181507, SLV-314, SLV-319, 7-OH-DPAT, VN-2222, PD-158771, RS-30199, WAY-100012, A-74283, Enilospirone, Org-13011, B-8805-033, AP-159, AZ-16596, Anpirtoline, Ebalzotan, Binospirone, MDL-72832, RU-24969, Bay-r-1531, Ipsapirone, BIMG 80, BMS-181100, BMS-181101, BMS-181970, BMY-7378, BW-1205U90, B-20991, HAT-90B, Nerisopam, LY-175644, LY-178210, LY-228729, LY-274600, LY-274601, LY-293284, LY-301317, LY-315535, E-4414, E-6265 citrate, Lesopitron, RGH-1756, RGH-1757, 1192U90, HP-236, FG-5938, LEK-8804, LB-50016, RWJ-25730, EMD-56551, EMD-67478, EMD-77697, Roxindole, Vilazodone, BP-554, CGP-50281, CGS-12066B, CGS-18102, SDZ-MAR-327, CL-870801, CP-110330, CP-146662, CP-291952, FCE-23892, FG-5865, FG-5893, OSU-191, Sunepitron, U-67413B, U-86170, U-86192A, U-92016A, U-93385, Eptapirone, Mazapertine succinate, SL-870765, SL-880338, SR-59026, Bromerguride, Alnespirone, S-14506, S-14671, S-15535, S-15931, S-16924, S-213571, S-215521, Elopiprazole, Eltoprazine, Flesinoxan, Umespirone, SUN-8399, S-23751, PM-1000, LY 41, Adatanserin, WY-48723, Zalospirone and MDL-73975, optionally in form of the pharmaceutically acceptable acid addition salts, in form of the hydrates and/or solvates and optionally in the form of the individual optical isomers, mixtures of the individual enantiomers or racemates thereof.

Preferred examples of suitable 5-HT-1A agonists include Urapidil, Buspirone, Aripiprazole, Ziprasidone, Naftopidil, Tandospirone, Nemonapride, Gepirone, Repinotan, Sumanirole, Xaliproden hydrochloride, Bifeprunox, AP-521, SUN-N4057, Sarizotan, MKC-242, OPC-14523, Eptapirone maleate, SLV-308, BTS-79018, R-137696, F-13640, SSR-181507, SLV-314, SLV-319, 7-OH-DPAT, VN-2222, PD-158771, RS-30199 and WAY-100012, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one dopamine agonist, optionally in combination with a pharmaceutically acceptable excipient.

Examples of suitable dopamine agonists include ABT-724, CP-226269, bromocriptin, cabergolin, alpha-dihydroergocryptin, lisuride, pergolide, pramipexol, roxindol, ropinirol, sopirinol, talipexol, bupropion and terguride optionally in form of the pharmaceutically acceptable acid addition salts, in form of the hydrates and/or solvates and optionally in the form of the individual optical isomers, mixtures of the individual enantiomers or racemates thereof.

Preferred examples of suitable dopamine agonist include pramipexol, bupropion roxindol, and talipexol, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one 5-HT2A/2C antagonist, optionally in combination with a pharmaceutically acceptable excipient. Examples of suitable 5-HT2A/2C antagonists include Aripiprazole, Fluoxetine, Nefazodone, Pizotifen, Risperidone, Sarpogrelate, Ziprasidone, Agomelatine, Asenapine, Eplivanserin, lloperidone, ketanserin, ritanserin, M 100907, Netamiftide, Ocaperidone, S-20098, Abaperidone, ACP-103, EMR 62218, LU-31-130, SL 650472, EGIS-10037, LEK-8829, Nantenine, QF-2004B, R-107500, S 35120, S-14297, Amesergide, Amperozide, AT 1015, Balaperidone, BIMG 80, Deramciclane, EGIS 8465, EGIS 9933, Fananserin, FG 5803, FG 5893, FG-5938, FG-5974, GMC 1169, GMC 283, GMC 306, GMC 6139, ICI-169369, Irindalone, IT 657, JL-13, Lubazodone, LY 215840, LY-367265, NRA-0045, Org-38457, PNU-96415E, QF 0510B, QF 1003B, QF 1004B, RO 600946, Ro-60-0759, RP 71602, RS-102221, S 16924, S 213571, S 35031, S-17828, S-21357-1, SB 200646A, SB 206553, SB 221284, SB 228357, SB 242084, SB 243213, SDZ SER 082, TY 12283, TY-11223 and ZD-3638 optionally in form of the pharmaceutically acceptable acid addition salts, in form of the hydrates and/or solvates and optionally in the form of the individual optical isomers, mixtures of the individual enantiomers or racemates thereof.

Preferred 5-HT2A/2C antagonist include Aripiprazole, Fluoxetine, Nefazodone, Pizotifen, Risperidone, Sarpogrelate, Ziprasidone, Agomelatine, Asenapine, Eplivanserin, Iloperidone, M 100907, Netamiftide, Ocaperidone, S-20098, Abaperidone, ketanserin, ritanserin, ACP-103, EMR 62218, LU-31-130, SL 650472, EGIS-10037, LEK-8829, Nantenine, QF-2004B, R-107500, S 35120 and S-14297 optionally in form of the pharmaceutically acceptable acid addition salts, in form of the hydrates and/or solvates and optionally in the form of the individual optical isomers, mixtures of the individual enantiomers or racemates thereof. Particular preferred 5-HT2A/2C antagonist are selected from among Aripiprazole, Fluoxetine, Nefazodone, Pizotifen, Risperidone, Sarpogrelate and Ziprasidone optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of 1 and a therapeutically effective amount of one or more, preferably one dopamine D4 antagonist, optionally in combination with a pharmaceutically acceptable excipient. Examples of suitable dopamine D4 antagonists include olanzapine, ziprasidone, MDL-814608A, NRA-0562, S-18126, SPI-376, YM-50001, 1192U90, ALX-D4, Balaperidone, BIMG 80, C₁₋₁₀₃₀, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, PD-089232, PD-108306, PD-165167, PD-167063, PD-168306, PD-172760, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, optionally in form of the pharmaceutically acceptable acid addition salts, in form of the hydrates and/or solvates and optionally in the form of the individual optical isomers, mixtures of the individual enantiomers or racemates thereof.

Preferred examples of suitable dopamine D4 antagonist include olanzapine, ziprasidone, MDL-814608A, NRA-0562, S-18126, SPI-376 and YM-50001, in particular olanzapine and ziprasidone, optionally in form of the pharmaceutically acceptable acid addition salts, in form of the hydrates and/or solvates and optionally in the form of the individual optical isomers, mixtures of the individual enantiomers or racemates thereof.

What is claimed is:

1) A method for the treatment of female sexual disorders comprising administration of therapeutically effective amount of a compound of formula 1 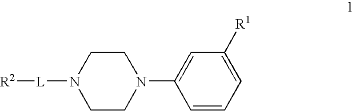
wherein
R¹ is a group selected from among halogen, —O—C₁-C₄-alkyl, and —C(halogen)₃;
L is a linker, selected from the bridging groups —C₁-C₆-alkylene, —C₁-C₄-alkylene-O—, —C₁-C₄-alkylene-O—CO—, —C₁-C₄-alkylene-N H—, —C₁-C₄-alkylene-NH—CO—, —C₂-C₆-alkenylene, —C₂-C₄-alkenylene-O—, —C₂-C₄-alkenylene-O—CO—, —C₂-C₄-alkenylene-NH—, —C₂-C₄-alkenylene-NH—CO—, —C₂-C₆-alkynylene, —C₂-C₄-alkynylene-O—, —C₂-C₄-alkynylene-O—CO—, —C₂-C₄-alkynylene-N H—, and
—C₂-C₄-alkynylene-NH—CO—, which may optionally be substituted by one or more, preferably one group selected from among —C₁-C₄-alkyl, —OH, halogen, ═O, —C(halogen)₃ and —O—C₁-C₄-alkyl;
R² is —NH₂, —NHC₁-C₄-alkyl, —N(C₁-C₄-alkyl)₂, or a group selected from among —C₁-C₆-alkyl and —C₃-C₆-cycloalkyl which may optionally be substituted by one or more, preferably one group selected from among —C₁-C₄-alkyl, —OH, halogen, ═O, —C(halogen)₃, —O—C₁-C₄-alkyl, —O—C₆-C₁₀-aryl, —NH₂, —NHC₁-C₄-alkyl, —N(C₁-C₄-alkyl)₂, —C₂-C₄-alkenyl and —C₂-C₄-alkynyl, or
R² is —C₆-C₁₀-aryl, optionally substituted by one or more, preferably one group selected from among, —C₁-C₄-alkyl, —OH, halogen, —C(halogen)₃, —O—C₁-C₄-alkyl, —NH₂, —NH—C₁-C₄-alkyl, —N(C₁-C₄-alkyl)₂, and a nitrogen containing heteroaromatic ring, wherein said nitrogen containing heteroaromatic ring may optionally be substituted by one or more, preferably one group selected from among —C₁-C₄-alkyl, —OH, halogen, —C(halogen)₃, and —O—C₁-C₄-alkyl, and wherein said nitrogen containing heteroaromatic ring my optionally be linked to the —C₆-C₁₀-aryl group via a bridging group selected from among —O—, —S—, and —NH—, or
R² is a group selected from among 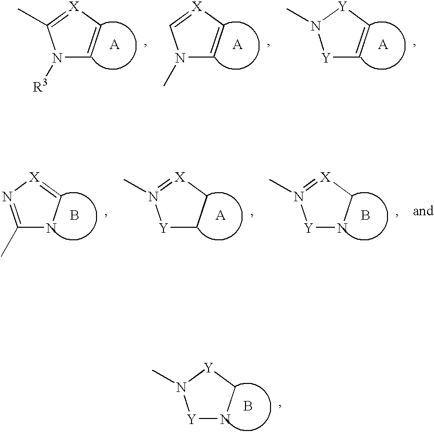
wherein
X is either N or —CR³—;
Y is either —NR⁵—, —O—, —S—, —SO₂—, —CH₂— or —CO—;
A is absent or a ring system selected from among 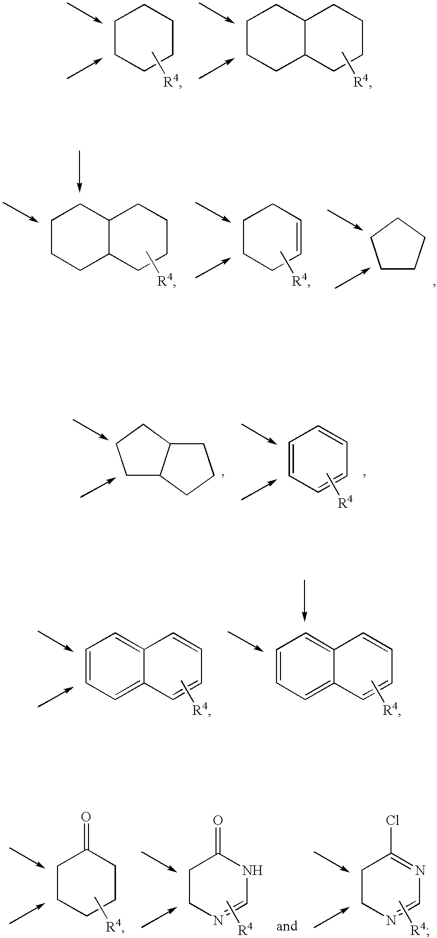
B is absent or a ring system selected from among 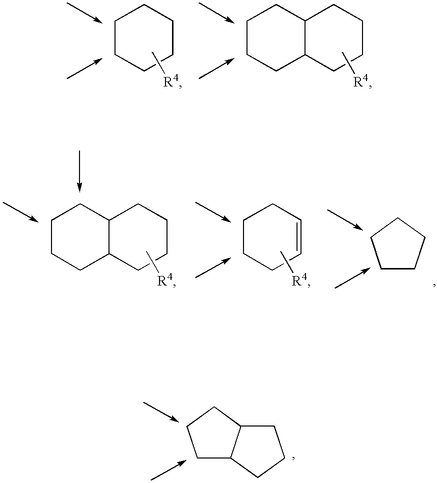
rein the arrows indicate the positions where the ring is annellated to the five membered nitrogen heterocycle, and wherein
R³ is selected from among hydrogen, —C₁-C₄-alkyl, —CH₂—NH₂, —CH₂—NH—C₁-C₄-alkyl, —CH₂—N(C₁-C₄-alkyl)₂, —NH₂, —NH—C₁-C₄-alkyl, and —N(C₁-C₄-alkyl)₂;
R⁴ is selected from among hydrogen, —C₁-C₄-alkyl, —OH, halogen, —C(halogen)₃ and —O—C₁-C₄-alkyl,
R⁵ is selected from among hydrogen, —C₁-C₄-alkyl, —C₆-C₁₀-aryl, and —C₁-C₄-alkylen-C₆-C₁₀-aryl;
a pharmaceutically acceptable acid addition salt thereof, a hydrate or solvate thereof, or in the form of the individual optical isomer, mixture of the individual enantiomers or a racemate thereof.

2) The method for the treatment of female sexual disorders according to claim 1, wherein the disorder is selected from the group consisting of Hypoactive Sexual Desire Disorder, loss of sexual desire, lack of sexual desire, decreased sexual desire, inhibited sexual desire, loss of libido, libido disturbance, and frigidity.

3) The method for the treatment of female sexual disorders according to claim 1, wherein the disorder is selected from premenstrual disorders.

4) The method for the treatment of female sexual disorders according to claim 1, wherein the disorder is sexual aversion disorder.

5) The method for the treatment of female sexual disorders according to claim 1, wherein the disorder is sexual arousal disorder.

6) The method for the treatment of female sexual disorders according to claim 1, wherein the disorder is orgasmic disorder.

7) The method for the treatment of female sexual disorders according to claim 1, wherein the disorder is a sexual pain disorder.

8) The method for the treatment of female sexual disorders according to claim 1, comprising administration of a therapeutically effective amount of a compound of formula 1, wherein
R¹ is a group selected from among fluorine, chlorine, —O-methyl, and —CF₃, preferably chlorine and —CF₃;
L is a linker, selected from the bridging groups —C₁-C₄-alkylene, —C₁-C₃-alkylene-O—, —C₁-C₃-alkylene-O—CO—, —C₁-C₃-alkylene-NH—, —C₁-C₃-alkylene-NH—CO—, and —C₂-C₄-alkenylene, which may optionally be substituted by one or more, preferably one group selected from among methyl, ethyl, propyl, —OH, chlorine, fluorine, =0 and —CF₃;
R² is —NH₂, —NHC₁-C₄-alkyl, —N(C₁-C₄-alkyl)₂, or a group selected from among —C₁-C₄-alkyl and —C₃-C₆-cycloalkyl which may optionally be substituted by one or more, preferably one group selected from among —OH, fluorine, chlorine, ═O, —CF₃, —O-phenyl, —O-naphthyl, —NH₂, —NHmethyl, —N(methyl)₂, —C₂-C₄-alkenyl and —C₂-C₄-alkynyl, or
R² is a phenyl or naphthyl group, optionally substituted by one or more, preferably one group selected from among, methyl, ethyl, —OH, fluorine, chlorine, —CF₃, —O-methyl, —O-ethyl, —NH₂,
—NH-methyl, —N(methyl)₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, ethyl, —OH, fluorine, chlorine, —CF₃, —O-methyl and —O-ethyl, and wherein said nitrogen containing heteroaromatic ring my optionally be linked to the phenyl or naphthyl group via a bridging group selected from among —O— and —NH—, or
R² is a group selected from among 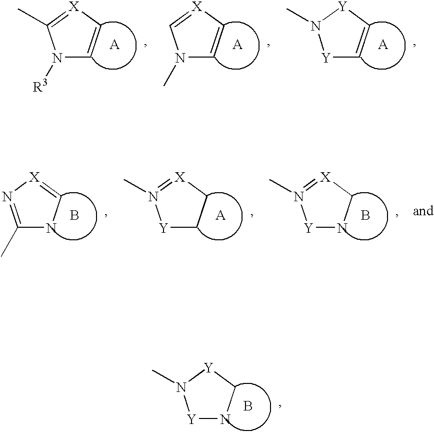
wherein
X is either N or —CR³—;
Y is either —NR⁵—, —O—, or —CO—;
A is absent or a ring system selected from among 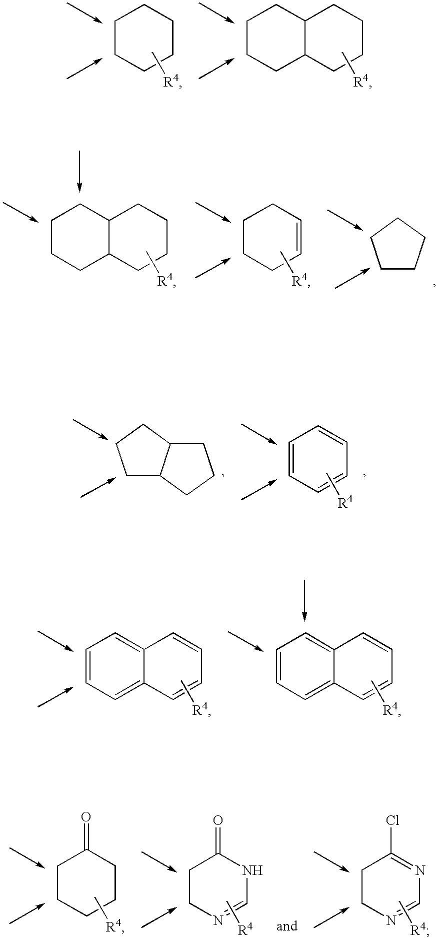
B is absent or a ring system selected from among 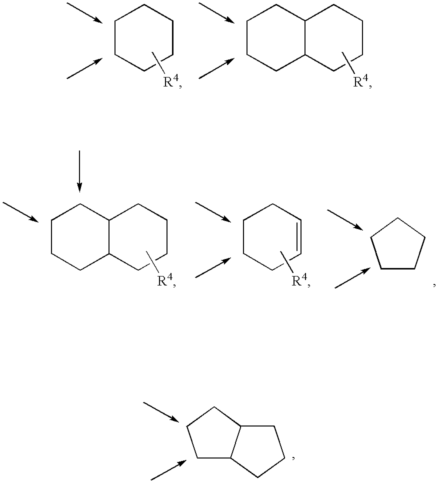
wherein the arrows indicate the positions where the ring is annellated to the five membered nitrogen heterocycle, and wherein
R³ is selected from among hydrogen, methyl, ethyl, propyl, —CH₂—N(methyl)₂, and —N(methyl)₂;
R⁴ is selected from among hydrogen, methyl, ethyl, propyl, —OH, chlorine, fluorine and —CF₃,
R⁵ is selected from among hydrogen, methyl, ethyl, propyl, phenyl, —CH₂—CH₂-phenyl and Benzyl;
a pharmaceutically acceptable acid addition salt thereof, a hydrate and/or solvate thereof, or in the form of the individual optical isomer, a mixture of the individual enantiomers or a racemate thereof.

9) The method for the treatment of female sexual disorders according to claim 1, comprising administration of a therapeutically effective amount of a compound of formula 1, wherein
R¹ is a group selected from among fluorine, chlorine, —O-methyl, and —CF₃, preferably chlorine and —CF₃;
L is a linker, selected from the bridging groups —C₁-C₄-alkylene, —C₁-C₃-alkylene-O—, —C₁-C₃-alkylene-O—CO—, —C₁-C₃-alkylene-NH—, —C₁-C₃-alkylene-NH—CO—, and —C₂-C₄-alkenylene, which may optionally be substituted by one or more, preferably one group selected from among methyl, ethyl, propyl, —OH, chlorine, fluorine, =0 and —CF₃;
R² is —NH₂, —NHC, —C₄-alkyl, —N(C₁-C₄-alkyl)₂, or a group selected from among —C₁-C₄-alkyl and —C₃-C₆-cycloalkyl which may optionally be substituted by one or more, preferably one group selected from among —OH, fluorine, chlorine, ═O, —CF₃, —O-phenyl, —O-naphthyl, —NH₂, —NHmethyl, —N(methyl)₂, —C₂-C₄-alkenyl and —C₂-C₄-alkynyl, or
R² is a phenyl or naphthyl group, optionally substituted by one or more, preferably one group selected from among, methyl, ethyl, —OH, fluorine, chlorine, —CF₃, —O-methyl, —O-ethyl, —NH₂,
—NH-methyl, —N(methyl)₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, ethyl, —OH, fluorine, chlorine, —CF₃, —O-methyl and —O-ethyl, and wherein said nitrogen containing heteroaromatic ring my optionally be linked to the phenyl or naphthyl group via a bridging group selected from among —O— and —NH—, or
R² is a group selected from among. 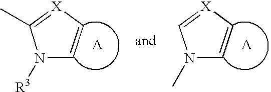
wherein
X is either N or —CR³—;
R³ is selected from among hydrogen, methyl, ethyl, propyl, —CH₂—N (methyl)₂, and —N(methyl)₂;
A is a ring system selected from among 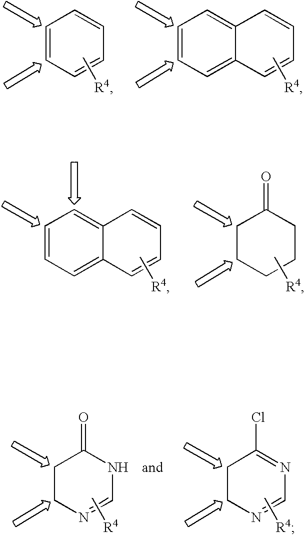
wherein the arrows indicate the positions where the ring is annellated to the five membered nitrogen heterocycle, and wherein
R⁴ is selected from among hydrogen, methyl, ethyl, propyl, —OH, chlorine, fluorine and —CF₃,
a pharmaceutically acceptable acid addition salt thereof, a hydrate and/or solvate thereof, or in the form of the individual optical isomer, a mixture of the individual enantiomers or a racemate thereof.

10) The method for the treatment of female sexual disorders according to claim 1, comprising administration of a therapeutically effective amount of a compound of formula 1, wherein
R¹ is a group selected from among chlorine and —CF₃, preferably —CF₃;
L is a linker, selected from —CH₂—CH₂—, —CH₂—CH₂—CH₂—, —CH₂—CH₂—CH₂—CH₂—, —O—CH₂—CH₂—, —O—CH₂—CH₂—CH₂—, —O—CH₂—CH₂—CH₂—CH₂—, —CO—O—CH₂—CH₂—, —CO—O—CH₂—CH₂—CH₂—, —CO—O—CH₂—CH₂—CH₂—CH₂—, —NH—CH₂—CH₂—, —N H—CH₂—CH₂—CH₂—, —N H—CH₂—CH₂—CH₂—CH₂—, —CO—N H—CH₂—CH₂—, —CO—NH—CH₂—CH₂—CH₂— and —CO—NH—CH₂—CH₂—CH₂—CH₂—, which may optionally be substituted by one or more, preferably one group selected from among methyl, —OH, fluorine, and —CF₃;
R² is —NH₂, —NHC₁-C₄-alkyl, —N(C₁-C₄-alkyl)₂, or a group selected from among methyl and ethyl which may optionally be substituted by one or more, preferably one group selected from among —OH, fluorine, chlorine, ═O, —CF₃, —O-phenyl, and —NH₂, or
R² is a group selected from among cyclopentyl and cyclohexyl, which may optionally be substituted by one or more, preferably one group selected from among —OH, fluorine, —CF₃, and —C≡C—, or
R² is a phenyl group, optionally substituted by one or more, preferably one group selected from among, methyl, —OH, fluorine, —CF₃, —NH₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, —OH, fluorine, and —CF₃, and wherein said nitrogen containing heteroaromatic ring my optionally be linked to the phenyl group via a bridging group selected from among —O— and —NH—, or
R² is a group selected from among 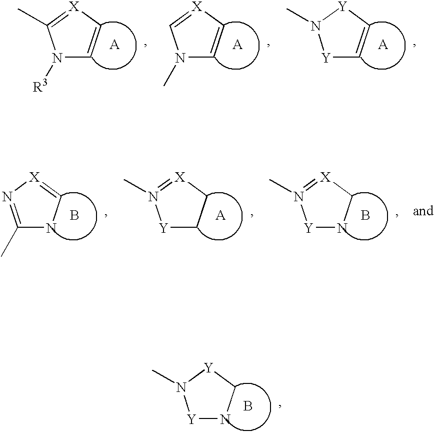
wherein
X is either N or —CH—;
Y is —O— or —CO—;
A is absent or a ring system selected from among 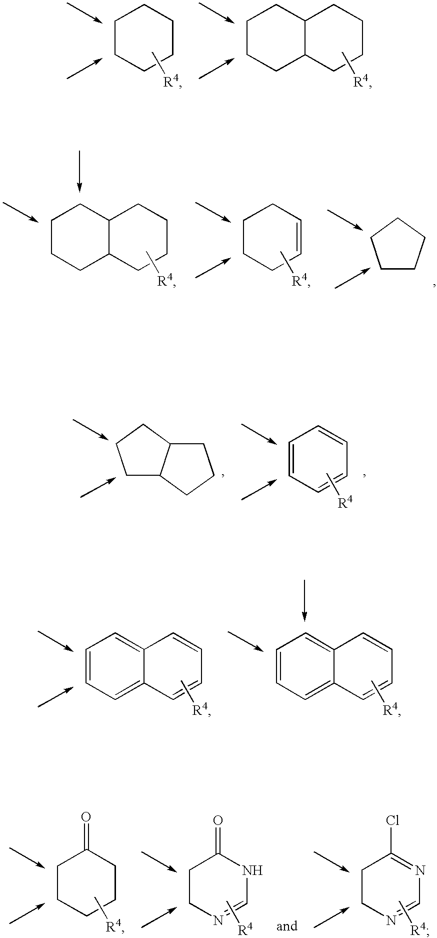
B is absent or a ring system selected from among 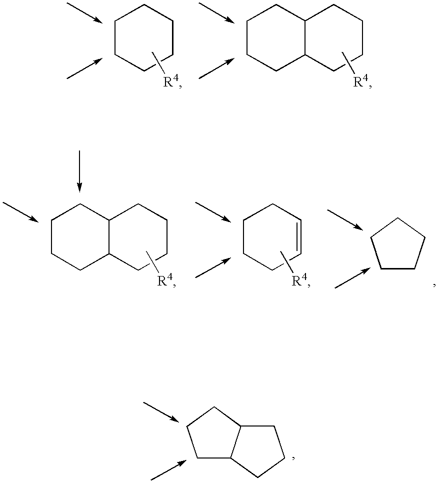
wherein the arrows indicate the positions where the ring is annellated to the five membered nitrogen heterocycle, and wherein
R⁴ is selected from among hydrogen, methyl, —OH, fluorine and —CF₃,
a pharmaceutically acceptable acid addition salt thereof, a hydrate and/or solvate or in the form of the individual optical isomer, a mixture of the individual enantiomers or a racemate thereof.

11) The method for the treatment of female sexual disorders according to claim 1, comprising administration of a therapeutically effective amount of a compound of formula 1, wherein
R¹ is a group selected from among chlorine and —CF₃, preferably —CF₃;
L is a linker, selected from —CH₂—CH₂—, —CH₂—CH₂—CH₂—, —CH₂—CH₂—CH₂—CH₂—, —O—CH₂—CH₂—, —O—CH₂—CH₂—CH₂—, —O—CH₂—CH₂—CH₂—CH₂—, —CO—O—CH₂—CH₂—, —CO—O—CH₂—CH₂—CH₂—, —CO—O—CH₂—CH₂—CH₂—CH₂—, —NH—CH₂—CH₂—, —NH—CH₂—CH₂—CH₂—, —NH—CH₂—CH₂—CH₂—CH₂—, —CO—NH—CH₂—CH₂—, —CO—NH—CH₂—CH₂—CH₂— and —CO—NH—CH₂—CH₂—CH₂—CH₂—, which may optionally be substituted by one or more, preferably one group selected from among methyl, —OH, fluorine, and —CF₃;
R² is —NH₂, —NHC₁-C₄-alkyl, —N(C₁-C₄-alkyl)₂, or a group selected from among methyl and ethyl which may optionally be substituted by one or more, preferably one group selected from among —OH, fluorine, chlorine, ═O, —CF₃, —O-phenyl, and —NH₂, or
R² is a group selected from among cyclopentyl and cyclohexyl, which may optionally be substituted by one or more, preferably one group selected from among —OH, fluorine, —CF₃, and —C≡C—, or
R² is a phenyl group, optionally substituted by one or more, preferably one group selected from among, methyl, —OH, fluorine, —CF₃, —NH₂, and a nitrogen containing heteroaromatic ring selected from among pyridine, pyrimidine, indol, pyrrole, imidazole, pyrazole, triazole, chinoline and isochinoline, wherein said nitrogen containing heteroaromatic ring may optionally be substituted by one or more, preferably one group selected from among methyl, —OH, fluorine, and —CF₃, and wherein said nitrogen containing heteroaromatic ring my optionally be linked to the phenyl group via a bridging group selected from among —O— and —NH—, or
R² is a group selected from among 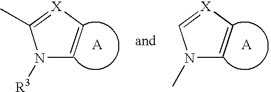
wherein
X is either N or —CH—;
R³ is selected from among hydrogen, isopropyl and —CH₂—N(methyl)₂;
A is a ring system selected from among 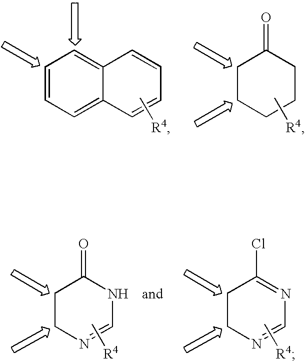
wherein the arrows indicate the positions where the ring is annellated to the five membered nitrogen heterocycle, and wherein
R⁴ is selected from among hydrogen, methyl, —OH, fluorine and —CF₃,
a pharmaceutically acceptable acid addition salt thereof, a the hydrate and/or solvate thereof, or in the form of the individual optical isomer, a mixture of the individual enantiomers or a racemate thereof.

12) A pharmaceutical composition for the treatment of female sexual disorders comprising a compound of formula 1 according to claim 1.

13) A pharmaceutical composition comprising a therapeutically effective amount of a compound of formula 1 according to claim 1 in combination with a therapeutically effective amount of one or more, preferably one active ingredient, preferably an active ingredient selected from the group consisting of melanocortin agonists, prostaglandin E1 agonists, elevators of cyclic guanosine 3′,5′-monophosphate (cGMP) (preferably PDE V inhibitors), 5-HT-1A agonists, dopamine agonists, dopamine D4 antagonist and 5-HT-2A/C antagonists.
